(19) United States
(12) Patent Application Publication
(10) Pub. No.: US 2022/0042119 A1
(43) Pub. Date: Feb. 10, 2022

(54) DETECTION OF DNA SEQUENCES AS RISK FACTORS FOR HIV INFECTION

(71) Applicant: Luc Montagnier, Le Plessis (FR)

(72) Inventor: Luc Montagnier, Le Plessis (FR)

(21) Appl. No.: 17/347,502

(22) Filed: Jun. 14, 2021

Related U.S. Application Data
(63) Continuation of application No. 16/299,107, filed on Mar. 11, 2019, now Pat. No. 11,035,011.
Application No. 16/299,107 is a continuation of application No. 14/851,837, filed on Sep. 11, 2015, now Pat. No. 10,227,665.
Application No. 14/851,837 is a continuation of application No. 13/752,003, filed on Jan. 28, 2013, now Pat. No. 9,133,525.
(60) Provisional application No. 61/716,123, filed on Oct. 19, 2012.

(51) Int. Cl. C12Q 1/70; C12Q 1/689; C12N 1/20; A61K 31/7052; A61K 31/65

(52) U.S. Cl. CPC C12Q 1/703; C12Q 1/689; C12N 15/11; A61K 31/7052; A61K 31/65; C12N 1/20

(57) ABSTRACT

A method for identifying a risk factor for diseases, disorders or conditions, such as those caused by human immunodeficiency virus, using the polymerase chain reaction and specific primers. Methods for treating patients having these diseases, disorders or conditions by antimicrobial treatment of the risk factor by combined antiviral and antibacterial treatment or by sustaining or stimulating the subject's immune system. Methods for screening biological products including red blood cell preparations. Primers and methods for detecting nucleic acids or microbial agents associated with red blood cells, such as those associated with red blood cells in subjects infected with HIV and undergoing antiretroviral therapy.

### CROSS-REFERENCE TO RELATED APPLICATIONS

This application is a:

Division of U.S. patent application Ser. No. 16/299,107, filed Mar. 11, 2019, now U.S. Pat. No. 11,035,011, issued Jun. 15, 2021, which is a

Division of U.S. patent application Ser. No. 14/851,837, filed Sep. 11, 2015, now U.S. Pat. No. 10,227,665, issued Mar. 12, 2019, which is a

Continuation of U.S. patent application Ser. No. 13/752,003, filed Jan. 28, 2013, now U.S. Pat. No. 9,133,525, issued Sep. 15, 2015, which

Claims benefit of priority under 35 U.S.C. § 119(e) to U.S. Provisional Application 61/716,123, filed Oct. 19, 2012 and

Claims benefit of priority under 35 U.S.C. § 119(e) to U.S. Provisional Application 61/591,111, filed Jan. 26, 2012,

each of which are each expressly incorporated herein by reference.

The subject matter disclosed in U.S. Application Nos. 61/186,610; 61/358,282; 61/476,110; 61/476,545; Ser. No. 12/797,286; 13/168,367; 61/591,111; and PCT/US2010/038160 is hereby expressly incorporated by reference in its entirety.

### BACKGROUND OF THE INVENTION

### Field of the Invention

DNA can be amplified from human red blood cell samples by primers designed from DNA sequences encoding a bacterial major surface protein and 16 s ribosomal RNA (16s rRNA). Primer pairs based on DNA sequences for the major surface protein 2 (MSP2) of Erhlichia/Anaplasma can amplify DNA homologous to DNA from human chromosomes 1 and 7 from red blood cell samples. Primers based on DNA sequences encoding 16s rRNA from Anaplasma species can amplify DNA from human red blood cells, but not from nucleated white blood cells. The amplified DNA is contained in samples of red blood cells from HIV infected individuals as well as from some healthy individuals of Caucasian or African origin and represents a risk factor for HIV infection. These primers can be employed in methods for assessing risk of HIV infection by amplifying DNA from red blood cell samples.

### Description of the Related Art

Chronic HIV infection causes strong immune depression (AIDS) in most patients leading to lethal opportunistic infections or cancers. Specific inhibitors of HIV multiplication are currently used for treating HIV infected patients before they reach the full-blown stage of AIDS.

Such inhibitors act mostly on the reverse transcriptase and protease of HIV to efficiently suppress virus multiplication and reduce virus load to a low level of less than 40 viral RNA copies per ml of blood. Treatment results in a partial recovery of the patient's immune system as evidenced by an increase of CD4 lymphocytes and reduction of or lessened severity of opportunistic infections. However, this treatment has to be given without interruption in order to prevent rebound of virus multiplication and a subsequent reduction in the numbers of CD4 lymphocytes. Rebound of viral infection is evidence of a reservoir of HIV in infected patients that is not accessible to antiviral treatment and the existence of this reservoir is generally acknowledged. In addition to a reservoir of HIV in infected patients, such patients often carry other microorganisms that are associated with HIV infection or that cause opportunistic infections.

Microorganisms associated with HIV infection that are detectable in human red blood cells, but not in human leukocytes or other kinds of nucleated human cells have not been previously characterized. The identification and characterization of microorganisms associated with HIV infection is of interest for purposes of assessing risk of HIV infection or determining the status of an HIV infected patient, for assessing risk or status of opportunistic infections, and to evaluate modes of treatment for HIV infected subjects.

### SUMMARY OF THE INVENTION

The primers designed and discovered by the inventor provide ways to pursue these objectives. Three kinds of primers have been developed and studied by the inventor.

The first kind of primer was designed based on the gene encoding the outer surface protein 2 of Ehrlichia/Anaplasma a genus of rickettsiales, which are known endosymbionts of other cells. These primers amplified DNA homologous to segments of DNA from human chromosomes 1 and 7. These primers are described in Appendix 2.

This first kind of primers were initially designed to detect DNA encoding the major surface protein 2 (MSP2) of Erhlichia/Anaplasma species. However, neither of the two pairs of primers described by Appendix 2 (Primer Pairs 1 and 2) detected at various annealing temperatures any related microorganism in the biological samples investigated.

Surprisingly, it was discovered that this first kind of primer amplified DNA from human red blood cell samples that was highly homologous to DNA sequences on segments of human chromosomes 1 and 7. Primer Pairs 1 and 2 amplified DNA by the polymerase chain reaction (“PCR”) that was 100% homologous with human sequences when the primer sequences themselves were excluded. The amplified DNA was sequenced and the sequences aligned to sequences described for human chromosome 1 (clone RP11-332J14 GI:22024579, clone RP11-410C4 GI:17985906, and Build GRCh37.p5 Primary Assembly-) and in human chromosome 7 (PAC clone RP4-728H9 GI:3980548; human Build GRCh37.p5, and alternate assembly HuRef SCAF_1103279188381:28934993-35424761). This was not expected since the primer pairs had been designed to detect genes encoding a bacterial MSP2 gene, not human chromosomal sequences. Furthermore, the amplification of such sequences from samples of red blood cells was in itself surprising since red blood cells lack a nucleus containing chromosomes. The ability to amplify DNA homologous to human DNA from red blood cells is evidence that the target DNA amplified by these primers is present as an extranuclear or cytoplasmic element, such as a plasmid, or is contained in or bound by a microorganism that invades or is otherwise associated with red blood cells. This DNA component may be present on a plasmid or otherwise contained in or bound to a microbe associated with red blood cells. Its presence represents a risk factor for HIV infection or progression and/or opportunistic infections.

A second kind of primer was designed based on the sequences homologous to human chromosomes 1 and 7 that were amplified by the first kind of primers (MSP2 primers). This kind of primer is useful for identifying the target DNA homologous to human chromosomes 1 and 7 in a sample, such as a red blood cell sample. Such primers, including the first type of MSP2 primers, are used to detect risks of HIV infection, HIV progression, risks of opportunistic infections, disease prognosis and response to drug treatment in subjects where the presence of DNA homologous to segments human chromosomes 1 and 7 is a risk factor. This kind of primer is exemplified in Appendix 3.

A third type of primer was developed based on the genes from Anaplasma species encoding 16s rRNA. Anaplasma is a genus of rickettsiales. This type of primer was found to amplify a sequence of 700 bp of ribosomal DNA that was about 85% identical to the corresponding genetic regions of Rickettsia and about 99% identical to the corresponding genetic regions of Acinetobacter genus. Acinetobacter is a genus of gram negative bacteria within the class of gammaproteobacteria. The homology of amplified 16s rDNA with Acinetobacter DNA may be coincidental because DNA can be amplified from biological samples that pass through a 450 nM filter unlike classical Acinetobacter.

These primers identify a bacterial agent associated with red blood cells that is related to but not identical to known Rickettsia species. This bacterial agent has been identified in red blood cells of not only HIV infected patients but also in some healthy individuals of Caucasian or African origin. This third type of primer is used to detect risks of HIV infection, HIV progression, risks of opportunistic infections, disease prognosis and response to drug treatment in subjects where the presence of a target containing the amplifiable 16s rRNA or 16s rDNA is a risk factor. These primers are described in Appendix 4.

### BRIEF DESCRIPTION OF THE DRAWINGS

FIGS. 1 and 2 show scans of gel electrophoresis lanes of different PCR amplicons.

### DETAILED DESCRIPTION OF THE INVENTION

Amplification of DNA from biological samples, including blood, plasma, and serum samples or samples obtained from cell culture can be performed using PCR or other nucleic amplification methods known in the art. These methods can be used to amplify or detect target DNA qualitatively or quantitatively to provide a “yes or no” determination of the presence of the target sequence or to quantitatively detect an amount of DNA amplified under controlled conditions.

The DNA amplified by the first and second kinds of primers is higher frequency in red blood cells obtained from patients infected with human immunodeficiency virus compared to healthy individuals. This is especially the case for patients who have undergone or are undergoing antiretroviral therapy. The quantity of DNA amplified by these primers is reduced after long-term treatment of a patient with antibiotics and the primers were found not to amplify DNA from white blood cells or from other human cell lines. DNA has also been amplified from the red blood cells of healthy African subjects not infected with HIV using these primers. These results suggest the amplified DNA is derived from an antibiotic sensitive microorganism associated with red blood cells.

Despite the apparent human origin of this DNA, the data herein show that the amplified sequences are associated with a transmissible agent and that the transmissible agent is closely associated with human immunodeficiency virus as explained below.

These sequences were easily detected in the DNA of anucleated red blood cells (RBCs) in 100% (35 out of 35 subjects) HIV-infected African and Caucasian patients and they could not be detected in the DNA of nucleated cells including in the white blood cell fraction of the same patients, nor in human DNA from cultured human cells.

These sequences were rarely detected in the red blood cell fraction of healthy African subjects and none were detected in the red blood cells of the healthy Caucasians tested.

Long term antibiotic treatment (e.g., with doxycycline or azithromycin) of HIV-positive patients for more than three months was found to decrease the intensity of the bands amplified using these primers suggesting that these bands are induced, generated or otherwise originate from an antibiotic sensitive microorganism.

Amplification of DNA from a supernatant of a short-term culture of human cell line HL60 with an extract of RBC from an HIV-positive patient prepared by freeze-thawing RBC and then by removing heavy components by a low speed (10 min. at 1,500 g) centrifugation produced strong DNA bands. The intensity of these bands suggests growth and multiplication of a microorganism that contains DNA amplified by Primer Pairs 1 and 2.

To further explore this effect, new primers were designed that allow amplification of regions of human genomic DNA adjacent to or including those amplified by Primer Pairs 1 and 2. These new primers include:

primers “hChr1114179308 S” upstream of, and “hChr1114179853 AS” encompassing one end of the 237 bp amplicon related to chromosome 1 (546-bp long amplicon);

primers “hChr7/4292976 S” upstream of, and “hChr7/4294619 AS” downstream of the 213 bp amplicon related to chromosome 7 (1,643-bp long amplicon); and the primers described by Appendix 3.

These primers amplify DNA not only from the components of RBCs of HIV infected Caucasian or African patients, but also from the components of RBCs of healthy African subjects. However, these DNAs are lacking in all or most of HIV-negative Caucasian subjects. These sequences are amplified after antibiotic treatment of their carrier subjects indicating that the agent generating them is insensitive to antibiotic treatment. As in the case of the MSP2 primers-amplified sequences, these kinds of primer pairs amplify DNA present in or associated with anucleated RBC and not in white blood cells or human cell lines. It is possible that such a microorganism identified with these primers differs from the carrier of the initial short DNA sequences and will amplify or cause amplification of human genomic DNA sequences in an integrated or unintegrated manner. The primers disclosed herein permit the design of diagnostic tests and treatments aimed at reducing the risk of HIV infection in important segments of the human population in which this agent appears and can be detected by amplification of these DNA sequences.

The third kind of primers that identify a previously unknown bacterial agent that is associated with human red blood cells and related to, but not identical to, known Rickettsia species are provided. These primers are derived from the 16S ribosomal DNA sequences of an Anaplasma species and amplify a sequence of 700 bp of ribosomal DNA that is about 89% identical to the corresponding regions of the genome of Rickettsia. This sequence is about 99% identical to the corresponding regions of Acinetobacter genus DNA. Besides the primers exemplified herein, other primers that amplify the same 700 bp of ribosomal DNA or detectable fragments of this sequences may be designed based on this nucleotide sequence. These primers may amplify 20, 30, 50, 100, 200, 300, 400, 500, 600 or 700 nucleotides of this sequence. They may comprise short portions (e.g., 18-30 bp) of the 700 bp sequence and can be designed based on methods well known in the molecular biological arts. The table below depicts the various kinds of primers.

Specific embodiments of the invention include, but are not limited to those described below.

An agent that is associated with red blood cells, especially mature anucleated red blood cells, that passes through a 0.45 micron filter. This agent may be sensitive or insensitive to a particular antibiotic. Agents sensitive to azithromycin or to a cyclin antibiotic have been identified. This agent contains, induces, excises, or otherwise provides DNA that is amplified by (i) primer pairs 1 (SEQ ID NOS: 3 and 4) or 2 (SEQ ID NOS: 5 and 6), (ii) a pair of primers described by Appendix 3 (SEQ ID NOS: 7-14 or SEQ ID NOS: 15-23), (iii), the pair of primers described in Appendix 4 (SEQ ID NOS: 24 and 25); or a pair primers that amplify at least fifteen, twenty, twenty five, thirty, forty, fifty or more consecutive nucleotides of the same DNA as is amplified by the specific primers described herein.

The amplified DNA may be 80%, 85%, 90%, 95%, 99%, up to and including 100% identical or similar to human DNA, wherein sequence identity is determined by BLASTn using the default setting. Preferred parameters for determining the “nucleotide identity” when using the BLASTN program (Altschul, S. et al., Journal of Molecular Biology 215 (1990), pages 403-410) are: Expect Threshold: 10; Word size: 28; Match Score: 1; Mismatch Score: −2; Gap costs: Linear.

An agent that is associated with red blood cells, passes through a 0.45 micron filter, may be sensitive or insensitive to a particular antibiotic, can be detectable in red blood cells of an HIV patient, but not detectable in the white blood cells of said patient. Such an agent may become detectable in the red blood cells of an HIV-infected patient within the first year after HIV infection or after initiation of anti-retroviral treatment. Such an agent may appear or be associated with the red blood cells of an African subject or European subject who is HIV-negative.

The agent may be a microorganism, such as a bacterium, or a specific kind of bacterium such as Rickettsia or Rickettsia-like bacteria, Ehrlichia or Anaplasma or a component thereof. Such an agent may contain a plasmid, episome, or extra chromosomal element comprising human chromosomal DNA that is amplified by MPS2 gene primers; or that is contained in or associated with a red blood cell that contains a plasmid or extra chromosomal element comprising human chromosomal DNA that is amplified by MPS2 gene primers.

Isolated red blood cells may contain the agent as described herein as well as disrupted or lysed red blood cells, such as a supernatant produced by freezing and thawing red blood cells after removing white blood cells and then removing material that pellets by a low speed centrifugation, e.g., for 10 min. at 1,500 g. The red blood cells associated with the agent are detected by amplifying DNA from them using (i) primer pairs 1 (SEQ ID NOS: 3 and 4) or 2 (SEQ ID NOS: 5 and 6), (ii) a pair of primers described by Appendix 3 (SEQ ID NOS: 7-14 or SEQ ID NOS: 15-23), (iii), the pair of primers described in Appendix 4 (SEQ ID NOS: 24 and 25); or a pair primers that amplify at least fifteen, twenty, twenty five, thirty, forty, fifty or more consecutive nucleotides of the same DNA as is amplified by the specific primers described herein.

Another aspect of the invention is the DNA amplified from the agent or from red blood cells associated with the agent. This DNA is can be produced using the primer pairs described herein. The DNA that is present in a red blood cell may be from an infectious or replicating agent per se, from a component of an infectious organism present in the anucleated red blood cell, or from DNA that results from exposure of the red blood cell or its precursor cells to an infectious or replicating agent.

The amplified DNA from a red blood cell may comprise portions of human chromosome 1 or 7 including the sequences described in Appendix 5 or Appendix 6 or fragments of these sequences comprising 10, 20, 30, 40, 50, 100, 200 or more consecutive nucleotides of these sequences.

The DNA according to the invention may be contained or inserted into a vector, such as a plasmid or phage vector containing the isolated or purified amplified DNA. A host cell can be transformed with the isolated or purified amplified DNA from the agent or from red blood cells associated with the agent.

The invention is also directed to a method for detecting an agent as described herein comprising contacting material from anucleated red blood cells of a subject with primer Pair 1, primer Pair 2, or a pair of primers selected from the group consisting of those described in Appendix 3 under conditions suitable for amplification of DNA by said primers, and detecting said agent when amplified DNA is detected.

The primers used in this method may be selected from the group consisting of (i) primer pairs 1 (SEQ ID NOS: 3 and 4) or 2 (SEQ ID NOS: 5 and 6), or (ii) a pair of primers described by Appendix 3 (SEQ ID NOS: 7-14 or SEQ ID NOS: 15-23) or a pair of primers that amplify at least fifteen, twenty, twenty five, thirty, forty, fifty or more consecutive nucleotides of the same DNA as is amplified by the specific primers described herein. Alternatively, a set of primers that amplify the same DNA fragment amplified by the two primers described above may be employed. These primers may be designed by methods known in the art and each may comprise 18-30 or more base pairs of the sequence amplified by the primers above.

A method for detecting an agent as described herein comprising: contacting under conditions suitable for amplification of target DNA material from red blood cells of a subject with a primer and detecting or recovering the amplified DNA, where the primers are described by Appendix 4:

Primer (sense)
(SEQ ID NO: 24)
5′-CTG ACG ACA GCC ATG CA
Primer (antisense)
(SEQ ID NO: 25)
5′-GCA GIG GGG AAT ATT GGA CA.

Alternatively, a set of primers that amplify the same DNA fragment amplified by the two primers described above may be employed. These primers may be designed by methods known in the art and each may comprise 18-30 or more base pairs of the sequence amplified by the two primers above.

The biological sample used in the method described above or other methods described herein may be whole blood or a cellular component of whole blood, isolated anucleated red blood cells, isolated red blood cell precursors, such as erythroblasts, bone marrow or spleen cells, or subcellular fractions thereof, such as cellular lysates, supernatants or solid materials. Blood plasma or serum or other bodily fluids or tissues may also be used as a biological sample for the methods described herein. Those of skill in the art can select an appropriate biological sample for performance of PCR or select the appropriate conditions for producing an EMS signalized sample based on the disclosures of the patent applications incorporated by reference above. Representative biological samples include whole blood, isolated RBCs, subcellular components, extracts, or lysates of RBCs or their precursor cells, blood plasma or blood serum, spinal fluid, mucosal secretions, urine, saliva, bone marrow, or tissues.

A method for treating or for reducing the severity of a disease, disorder, or condition associated with the agent comprising treating a patient with an agent that reduces the titer of said agent or that reduces the amount of DNA amplified from a cell associated with it. This method may also comprise treating the patient with one or more antibiotics, such as azithromycin or a cyclin antibiotic; with one or more synthetic or natural immunostimulants, active vaccines, passive vaccines, antioxidants or antibiotics. A patient may also undergo treatment sequentially or simultaneously for viruses or other microorganisms or agents capable of causing an immunodeficient disease, disorder or condition. Treatment may be therapeutic or prophylactic and can include the administration of one or more anti-retroviral drugs or other antiretroviral treatments. The patient may be currently undergoing antiretroviral therapy or therapy to eradicate human immunodeficiency virus infection and treatment for the coinfecting bacterium initiated. Other modes of or supplemental treatments include treating the patient with one or more natural immuno stimulants, antioxidants or antibiotics.

The methods described herein may employ samples from subjects or patients of different geographic origins or racial or genetic backgrounds. A subject or patient may be HIV-negative, recently (e.g., less than one year) HIV-positive, a patient who has been HIV-position for more than one or two years, an HIV-positive patient who has undergone or is undergoing anti-retroviral treatments or other kinds of patients who are HIV-positive such as those with AIDS or subjects at risk of becoming HIV-positive, developing AIDS or opportunistic infections. Patients may be of African origin or may have lived in Africa and exposed to biological and environmental agents there. Similarly, a patient may be of European or Caucasian origin or may have lived in Europe or America and exposed to biological and environmental agents there.

The invention is also directed to a method for treating a disease, disorder or condition associated with an agent described herein comprising contacting red blood cells with a substance that reduces the amount of DNA amplified from a red blood cell using (i) Primer Pairs 1 or 2, (ii) primers described by Appendix 3, (iii) or the primers described in Appendix 4 or primer pairs that amplify at least 20 consecutive nucleotides of the amplicons amplified by the primer pairs described above. Such a method for treating a disease, disorder or condition associated with an agent described herein may comprise contacting red blood cells of a subject with a substance that reduces the transmission of said agent to the red blood cell; may comprise replacing the red blood cells in a subject with red blood cells that are not associated with said agent or by stimulating the development of new red blood cells in said subject; or may comprise treating blood or red blood cells with an agent that that degrades, crosslinks or otherwise interferes or inactivates nucleic acids inside of or associated with a red blood cell.

Another aspect of the invention is a method for screening blood for red blood cells from which DNA can be amplified using (i) primer pairs 1 (SEQ ID NOS: 3 and 4) or 2 (SEQ ID NOS: 5 and 6), (ii) a pair of primers described by Appendix 3 (SEQ ID NOS: 7-14 or SEQ ID NOS: 15-23), (iii), the pair of primers described in Appendix 4 (SEQ ID NOS: 24 and 25); or a pair primers that amplify at least fifteen, twenty, twenty five, thirty, forty, fifty or more consecutive nucleotides of the same DNA as is amplified by the specific primers described herein. This method comprises contacting a sample of blood or red blood cells with these pairs of primers and detecting amplified DNA and selecting a blood sample from which DNA was amplified or alternatively selecting a blood sample from which no DNA was amplified. For example, a blood sample from which amplified DNA is detected may be further evaluated or cultured to determine the sensitivity of the red blood cells or the agent associated with them to antibiotic or other therapeutic treatments. Alternatively, a blood sample in from which no DNA is amplified may be assessed as being free of the agent associated with the DNA amplified by these primers.

### Example 1. Detection of Amplified DNA in Red Blood Cells

Separation of Red Blood Cells

Standard procedures for separating RBCs from buffy coat and other peripheral blood components are known. Peripheral blood was processed on a Ficoll gradient to separate the buffy coat from red blood cells. After such separation it was found that DNA extracted from buffy coat cells was completely negative as determined by PCR using the primers described above while the same primers amplified DNA in the fraction containing the separated red blood cells. While it cannot be ruled out that the agent detected is externally associated with the red blood cell membranes, it was found that amplified DNA was only detected in a supernatant prepared by a low speed (1,500 g×10 min.) centrifugation to remove the heavy components of a red blood cell lysate. This lysate was prepared by repeated freeze-thawing of red blood cells isolated from the buffy coat, strong shaking by vortex, and a low speed centrifugation (1,500 g×10 min.). A pellet and supernatant fraction were obtained and tested. The primers described above only amplified DNA in the supernatant fraction, but not in the pellet.

Growth on HL-60 Cells

HL-60 cells are an ATCC cell line of promyelocytic origin. Samples of HL-60 cells at a density of 5×105 cells per ml in RPMI medium supplemented with 10% fetal calf serum were inoculated with the supernatant of the red blood cell lysate described above. This lysate was obtained from the red blood cells of HIV-positive patients after freezing, thawing and vortexing as previously described. After culturing for 3 days at 37° C. the low speed (1,500 g×10 mins) supernatants of the cultures were tested by PCR for DNA amplified using Primer Pairs 1 and 2. DNA was amplified from all of these cultures up to a dilution of 10-8. The same results were obtained from culture supernatant that was passaged through a 0.45 micron filter.

Effects of Long-Term Antibiotic Treatment

Five HIV-positive patients were maintained on their antiretroviral therapy, but received for at least three months a daily antibiotic treatment (azithromycin 250 mg/day or doxycycline 100 mg/day). Blood samples were fractionated to recover a red blood cell fraction on day 0 and after 3 months of antibiotic. Results indicated that the amount of DNA amplified after 3 months of antibiotic treatment was significantly less than that amplified under the same conditions from the samples obtained on day 0.

Detection of Amplified DNA in Red Blood Cells of African and Caucasian Patients

Blood samples were obtained from African and European Patients who were HIV-negative or HIV-positive. Red blood cells were isolated from buffy coat and other blood components by separation on a Ficoll gradient as described above. Table 1 shows the results of amplification of red blood cell samples from these patients using Primer Pairs 1 and 2. Similar results were obtained using the primers described in Appendix 3. No DNA was amplified using Primer Pairs 3 and 4 for Chromosome 1 and 7 from the red blood cells of one European patient who was HIV-positive for a year or less. This suggests that in some Caucasians that the accumulation of this human DNA in the red blood cell fraction occurs late after infection and possibly under the selective pressure of antiretroviral treatment. However, amplified DNA was detected in this patient using the Primer Pairs 1 and 2 shown in Appendix 2, but the amplified DNA bands were weaker than those for chronically-infected HIV-positive patients.

TABLE 1

| | | | | | Caucasian | | | | Cultured cells | |
|---|---|---|---|---|---|---|---|---|---|---|
| | | | | | | | | RBC: | (HL60) | |
| | African | | | | | | | HIV+ | | HLCO + |
| | | | | RBC: | | | | treated | | RBC |
| | | | | HIV+ | | | | with | | extract |
| | | | | treated | | | | antibiotics | | from |
| | | | | with | | | | for 3 | | HIV+ |
| | | | | antibiotics | | | | months | | subject |
| | RBC: | RBC: | WBC: | for 3 | RBC: | RBC: | WBC: | (0 vs 3 | NO | (0 vs 3 |
| | HIV− | HIV+ | HIV+ | months | HIV− | HIV+ | HIV+ | months) | extract | days) |
| App.2 | | | | | | | | | | |
| Pair 1 | rare | 100% | 0% | ↓ | 0% | 100% | 0% | ↓ | − | ↑ |
| Pair 2 | rare | 100% | 0% | ↓ | 0% | 100% | 0% | ↓ | − | ↑ |
| Chr 1 | | | | | | | | | | |
| Pair 1 | + | + | − | + | − | + or − * | | + | − | |
| Chr 7 | | | | | | | | | | |
| Pair 1 | + | + | − | + | − | + or − * | | + | − | |

+ in chronically infected and treated patients
− in recently infected patients

DNA Amplified Using Primer Pairs 1 and 2

MSP2 Primer Pairs 1 and 2 were used to perform PCR on red blood cells of HIV-positive subjects are removal of white blood cells and other blood components by Ficoll gradient separation. Primer Pairs 1 and 2 are shown below.

Primer Pair 1
18 mer
(SEQ ID NO. 3)
5′ GCCTA CAGAT TAAAG GCT
20 mer
(SEQ ID NO. 4)
5′ ATCAT ARTCA CCATC ACCTA
Primer Pair 2:
17 mer
(SEQ ID NO. 5)
5′ CYTAC AGAGT GAAGG CT
20 mer
(SEQ ID NO. 6)
5′ ATCAT ARTCA CCATC ACCTA

The DNA bands amplified by PCR using Primer Pairs 1 and 2 were 100% homologous with human sequences (primer sequences excluded) present in data-banks for human genomic sequences, respectively in human chromosome 1 (clone RPII-332J14 GI:22024579, clone RP11-410C4 GI:17985906, and Build GRCh37.p5 Primary Assembly-) and in human chromosome 7 (PAC clone RP4-728H9 GI:3980548; human Build GRCh37.p5, and alternate assembly HuRefSCAF_1103279188381:28934993-35424761).

Human Chromosome 1 and 7 DNA Sequences Described in Appendix 5

Appendix 5 shows the identities of human chromosome 1 and Chromosome 7 sequences that are amplified by primers described in Appendix 3. Primer sequences are underlined.

Primer Pair 3: Primer “hChr1114179308 S” upstream of, and “hChr1/14179853 AS” encompassing one end of the 237 bp amplicon related to chromosome 1 (546-bp long amplicon) were used to perform PCR on material from red blood cells isolated from other blood components by Ficoll gradient.

Primer Pair 4: Primers “hChr7/4292976 S” upstream of, and “hChr7/4294619 AS” downstream of the 213 bp amplicon related to chromosome 7 (1,643-bp long amplicon); and the primers described by Appendix 3.

### Example 2. Method for Detecting Risk of Acquiring HIV Infection or Opportunistic Infection Associated with HIV Infection or Risk of the Progression of an HIV Infection or Opportunistic Infection

Blood is collected from a subject in the presence of EDTA as an anticoagulant. The red blood cells in the sample are separated from buffy coat and plasma components of blood using a Ficoll-Hypaque gradient according to the manufacturer's current protocol. DNA in the red blood cell sample is prepared and amplified using a QIAGEN® Fast Cycling PCR Kit or Taq PCR Core Kit (as described in the current QIAGEN® product catalog) using MSP2 primer pair 1:

(SEQ ID NO: 3)
5′ GCCTA CAGAT TAAAG GCT
and
(SEQ ID NO: 4)
5′ ATCAT ARTCA CCATC ACCTA
or
MSP2 primer pair 2:
(SEQ ID NO: 5)
5′ CYTAC AGAGT GAAGG CT
and
(SEQ ID NO: 6)
5′ ATCAT ARTCA CCATC ACCTA.

Amplified DNA is resolved by gel electrophoresis and detected by staining with ethidium bromide. A subject is classified as being at a higher risk for acquiring HIV or HIV-associated opportunistic infection or for when amplified DNA is detected.

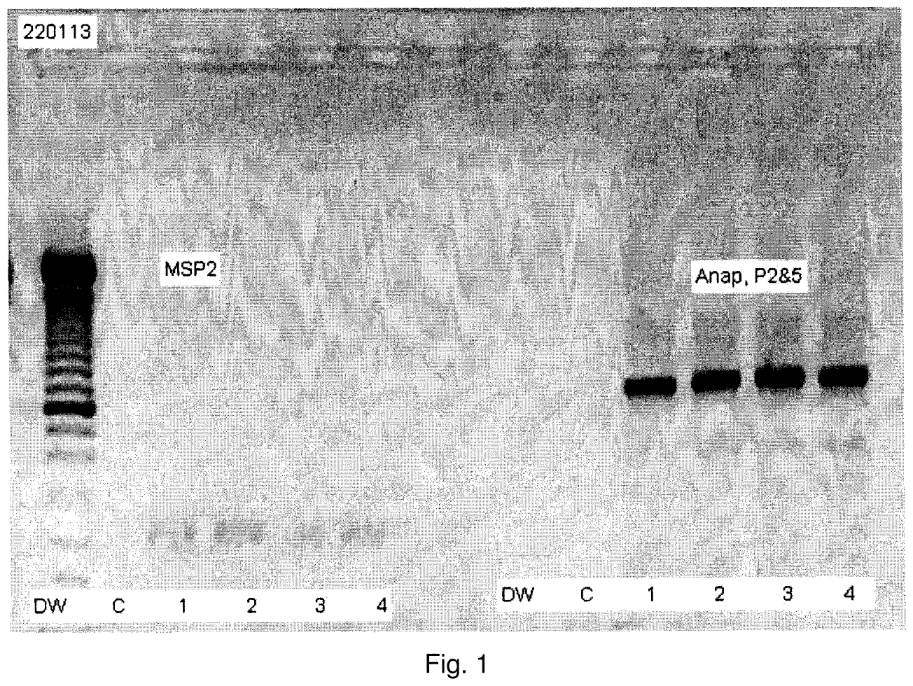

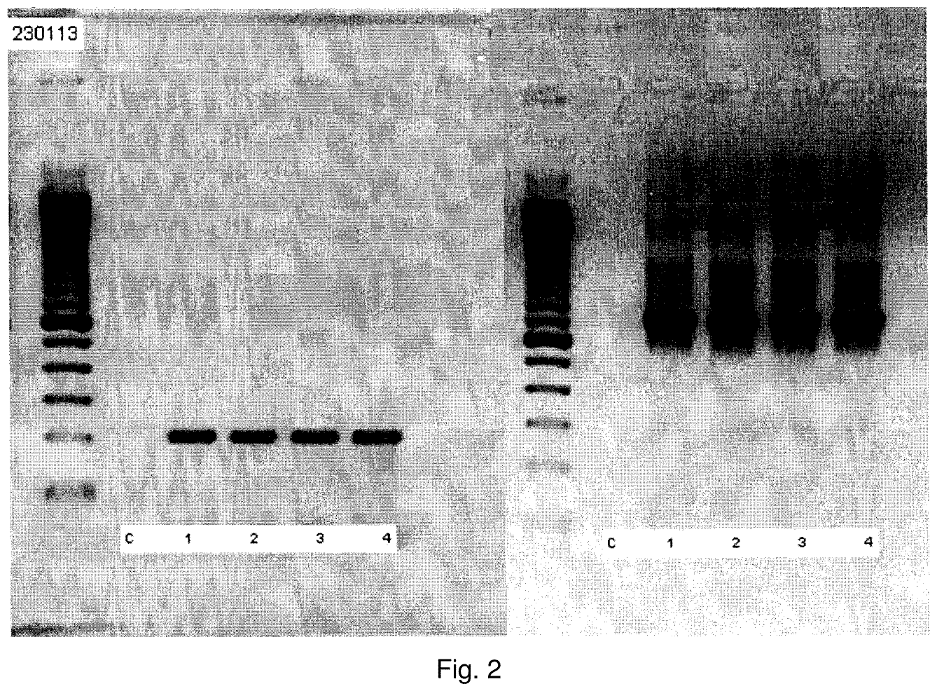

FIGS. 1 and 2 were produced on different dates. Lane numbers 1 and 2 are duplicates (from HIV-negative source) and lanes 3 and 4 are duplicates (from HIV-positive source). All DNA samples were extracted from a third passage HL60 cells exposed to an agent originating from red blood cells of HIV-negative or HIV-positive patients. These panels represent gel electrophoresis pictures of amplicons obtained by 70 cycles of PCR using primers derived from the Ehrlichia MSP2 gene (213 bp) and primers derived from the 16s ribosomal gene of Anaplasma (690 bp). The bands on the left sides of FIGS. 1 (A) and 1(B) show the 213 bp fragment mapped to human chromosome 7 amplified by the Ehrlichia MSP2 primers. The bands on the right sides of FIGS. 1 and 2 show the 690 bp fragment amplified by the Anaplasma primers (SEQ ID NOS: 24 and 25).

### Example 3. Method for Detecting Microorganism Associated with Risk or Progression of HIV Infection or Opportunistic Infection Associated with Infection with HIV

Blood is collected from a subject in the presence of EDTA as an anticoagulant. The red blood cells in the sample are separated from buffy coat and plasma components of blood using a Ficoll-Hypaque gradient according to the manufacturer's current protocol. DNA in the red blood cell sample is prepared and amplified using a QIAGEN® Fast Cycling PCR Kit or Taq PCR Core Kit (as described in the current QIAGEN® product catalog) using the primer pair

5′-CTG ACG ACA GCC ATG CA (SEQ ID NO: 24) and

5′-GCA GTG GGG AAT ATT GGA CA (SEQ ID NO: 25).

Amplified DNA is resolved by gel electrophoresis and detected by staining with ethidium bromide. A subject is identified as being infected with a microorganism when amplified DNA is detected.

APPENDIX 1

| | | Primers were |
|---|---|---|
| | | designed to |
| | | include a |
| | | conserved 16S |
| Primers | | rickettsiales |
| designed | | 16S region of |
| based on | | DNA which |
| Rickettsiales | | ribosomal |
| 16s | | DNA recognizes |
| ribosomal | SEQ | Rickettsiales |
| DNA | ID | and also |
| gene | NO: | Propionobacter |
| 5′-GCAAGCGG | SEQ | Rick 16S 929S |
| AAAAACCTTA | ID | (20 mer) |
| CC | NO: | Tm = 45.0° C. |
| | 1 | |
| 5′-GACGGGCA | SEQ | Rick 16S 1373AS |
| GTGTGTACAA | ID | (18 mer) |
| | NO: | Tm = 45.0° C. |
| | 2 | |

APPENDIX 2

| | MSP2 | | |
|---|---|---|---|
| | Primers | SEQ ID NO: | |
| MSP primer | 5′-GCCTAC | SEQ ID | 18-mer |
| pair 1 | AGATTAAAG | NO: 3 | |
| | GCT | | |
| | 5′-ATCATA | SEQ ID | 20-mer |
| | RTCACCATC | NO: 4 | |
| | ACCTA | | |
| MSP primer | 5′-CYTACA | SEQ ID | 17-mer |
| pair 2 | GAGTGAAGG | NO: 5 | |
| | CT | | |
| | 5′-ATCATAR | SEQ ID | 20-mer |
| | TCACCATCAC | NO: 6 | |
| | CTA | | |

APPENDIX 3
Human chromosome 1 and 7 primers

| Chromosome 1 | | | |
|---|---|---|---|
| primers | | SEQ ID NO: | |
| Primer #1 | 5′-C | SEQ ID | hChr1/14179308 S |
| | CTTA | NO: 7 | |
| | CACT | | |
| | CAGC | | |
| | CAGA | | |
| | CAT | | |
| Primer #2 | 5′-C | SEQ ID | hChr1/14179853 AS |
| | CAGG | NO: 8 | |
| | TGTG | | |
| | TGGC | | |
| | TTAT | | |
| | ACA | | |
| Primer #3 | 5′-C | SEQ ID | hChr1/14180006 S |
| | ATAG | NO: 9 | |
| | CTTC | | |
| | CTAG | | |
| | TAAG | | |
| | TAGA | | |
| | CCAG | | |
| Primer #4 | 5′-A | SEQ ID | hChr1/14180401 AS |
| | GGGG | NO: 10 | |
| | AGTC | | |
| | TGAG | | |
| | ATGG | | |
| | T | | |
| Primer #5 | 5′-A | SEQ ID | hChr1/14181093 AS |
| | CTGG | NO: 11 | |
| | AGAG | | |
| | GTGG | | |
| | AGGT | | |
| | T | | |
| Primer #6 | 5′-G | SEQ ID | hChr1/14181390 AS |
| | GTAA | NO: 12 | |
| | TTCC | | |
| | TATG | | |
| | TGCG | | |
| | AGGT | | |
| Primer #7 | 5′-T | SEQ ID | hChr1/14177990 S |
| | CAAA | NO: 13 | |
| | AGAC | | |
| | AGTG | | |
| | GTGA | | |
| | CTCT | | |
| Primer #8 | 5′-A | SEQ ID | hChr1/14179327 AS |
| | TGTC | NO: 14 | |
| | TGGC | | |
| | TGAG | | |
| | TGTA | | |
| | AGG | | |
| Chromosome 7 | | | |
| primers | | SEQ ID NO: | |
| Primer #1 | 5′-A | SEQ ID | hChr7/4292976 S |
| | TGTA | NO: 15 | |
| | GTTG | | |
| | AGCA | | |
| | GTTT | | |
| | TGAA | | |
| | TGA | | |
| Primer #2 | 5′-T | SEQ ID | hChr7/4293490 S |
| | CCTG | NO: 16 | |
| | CCTT | | |
| | AGTG | | |
| | AGGA | | |
| | TCT | | |
| Primer #3 | 5′-A | SEQ ID | hChr7/4293941 AS |
| | TGAA | NO: 17 | |
| | TAGG | | |
| | TGAT | | |
| | GGTG | | |
| | ATGA | | |
| | CT | | |
| Primer #4 | 5′-C | SEQ ID | hChr7/4294102 S |
| | CGTC | NO: 18 | |
| | ATTT | | |
| | AAGC | | |
| | CTTT | | |
| | AATC | | |
| | TCA | | |
| Primer #5 | 5′-G | SEQ ID | hChr7/4294619 AS |
| | TAGT | NO: 19 | |
| | CTTT | | |
| | TGGC | | |
| | ATCT | | |
| | CTTT | | |
| | GTA | | |
| Primer #6 | 5′-C | SEQ ID | hChr7/4294983 AS |
| | AGCC | NO: 20 | |
| | TGGA | | |
| | GAAC | | |
| | AGAG | | |
| | TG | | |
| Primer #7 | 5′-G | SEQ ID | hChr7/4295251 AS |
| | ATCT | NO: 21 | |
| | AGCA | | |
| | GTTC | | |
| | ATAG | | |
| | GAAG | | |
| | GAA | | |
| Primer #8 | 5′-G | SEQ ID | hChr7/4291579 S |
| | GCTG | NO: 22 | |
| | AAAC | | |
| | AATG | | |
| | GGGT | | |
| | TTT | | |
| Primer #9 | 5′-T | SEQ ID | hChr7/4293175 AS |
| | TTAG | NO: 23 | |
| | TAGA | | |
| | CCCC | | |
| | TCGA | | |
| | CCA | | |

APPENDIX 4
Anaplasma 16s rDNA based primers

| | | SEQ ID NO: | |
|---|---|---|---|
| Primer | 5′-CTGACGACA | SEQ ID NO: 24 | Sense |
| | GCCATGCA | | |
| Primer | 5′-GCAGTGGGG | SEQ ID NO: 25 | antisense |
| | AATATTGGACA | | |

APPENDIX 5
The human chromosome 1 sequence amplified by the MSP2
primers shown below:

| | primer identifiers | Sequence |
|---|---|---|
| MSP2 primer #3) | Ac/mMSP2-10195 | 5′-CYTACAGAGTGAAGGCT |
| | | (SEQ ID NO: 5) |
| MSP2 primer #5) | Ac/mMSP2-1128A5 | 5′-ATCATARTCACCATCACC |
| | | TA |
| | | (SEQ ID NO: 6) |

Y = T or C; R = G or A
Sequence of the 237 bp amplicon (SEQ ID NO: 26)
generated with these two primers by PCR:
5′-
ATCATAGTCA CCATCACCTA CCAGCTGTAT AAGCCACACA CCTGGGAGTC
CTCCTAGCCT TTTTCCTCCT CCTCTCATCC TCCATATCCC ATTGACCGTC
AGGGCCTACT GAGTCTACAC TCCAATTTTC TTTTAAATCT ATCCCCACTG
CCACTGTCCT AGTCTAAGGC AATACCATCT GGTCACCCAG ATCATTCCAT
AGCTTCCTAG TAAGTAGACC AGCCTTCACT CTGTAAG-3′
underlined nucleotides: primer sequence.
bold nucleotides: divergent nucleotides between MSP2
primers and the corresponding homologous human Chr 1
sequence.
The human chromosome 7 sequence amplified by the MSP2
primers shown below:

| | Primer identifiers | Sequence |
|---|---|---|
| MSP2 primer #2) | AphMSP2-10195 | 5′-GCCTACAGATTAAAGGCT |
| | | (SEQ ID NO: 3) |
| MSP2 primer #5) | Ac/mMSP2-1128A5 | 5′-ATCATARTCACCATCACC |
| | | TA |
| | | (SEQ ID NO: 6) |

Y = T or C; R = G or A
Sequence of the 213 bp amplicon (SEQ ID NO: 27) generated
with these two primers by PCR:
5′-
GCCTACAGAT TAAAGGCTTA AATGACGGTG AAAACTTAGT ATTCTTTGGG
TGGACAATAG TGAAATTTGC ACTTTGGACA GAATGACATG TACAAAAAGA
GTCAAGAAAC TTTTTAATCT ATTTAAAGGA CTCAAAGTAA TTTGTGAAGG
CCATAGCGTA AAATAACTTC AGTGGATGGA ATGGGATGAT GAATAGGTGA
TGGTGACTAT GAT-3′
underlined nucleotides: primer sequence.
bold nucleotides: divergent nucleotides between MSP2
primersand the corresponding homologous human Chr 7
sequence.
Identities of the human sequences amplified by the
human chromosome 1 primers or human chromosome 7
primers described in sections A) and B)
respectively below. The sequences described below,
except for the primers MSP2-derived amplicons, are
corresponding to human genetic sequences available
in NCBI genome databanks
(www.ncbi.nlm.nih.gov/projects/genome).
A) Genomic human Chromosome 1 primers #1
(hChr1/14179308 S) and #2 (hChr1/14179853 AS)
PCR-amplified 546 bp amplicon (SEQ ID NO: 28):
identifier: Amplicon hChr1/14179308-14179853
5′-
CCTTACACTC AGCCAGACAT ATATTTGTGT TTTGTTATCC ATGTGCACAG
AGACTTTGGC ATTCTGGGTG AAGGAAGAAA GAAGAGAATA TACATGGAAA
CCCAGGGGTA AGAGAAAAGG ACAACAGAGA ATGTGGCATG GGGAATGCTC
TGCTGGGTCA CATTGAATGG TTCTGAACCA CTGTGGAAAA AAAGGAGTTA
GAAAGAATCA GATGCCGAAG GAGCCAATTT TCACAATACT CCGAGACTCA
GGGCAAAAGC AGCCTTGTTC TAGTAGCCTA TGGGTAAAAG AAGACACAGA
ACTGAGGGGA GGACTTTTCC CCTGAGTCCA CCACAAACCG CCATGGAGCT
GAGGCAGCCT GAAGTCTCAG GGGCATGGGA GGGATTTGCC TTTTGGATTT
CTCCAATGGG ATGTCTTACA GGCACTTCAT ATTTAGCAGA TCCAAAACTT
AACTCAGATA CTCCTCTTGC CATATCTGTT CCTCTTGCTG TGTTCCTGAC
CATGATTATC ACCATCACCT ACCAGCTGTA TAAGCCACAC ACCTGG-3′
underlined nucleotides: hChr1 genomic primer sequence.
bold nucleotides: homologous extremity of the 237 bp
amplicon obtained with the primers MSP2#3-5.
B) Genomic human Chromosome 7 primers
#1 (hChr7/4292976 S) and #5
(hChr7/4294619 AS) PCR-amplified 1,
644 bp amplicon (SEQ ID NO: 29):
Identifier: Amplicon hChr7/4292976-4294619
5′-
ATGTAGTTGA GCAGTTTTGA ATGAGTTTCT TAATCCTGAG TTCTAGTTTA
AGAAAATATT AAAAATAAAA AATTATGTCA CCAACTAAAT TTTTACTGCA
GATAATCATA AGTTGGTTAG ATTGGACCTT CATTGTGAAA TGCAGTAACT
TTGGTTTAAG CAATATCCAA AACCAGAAAT TGGTCGAGGG GTCTACTAAA
TTCCGTTTTC TTTTGTTCTA AACAATTAAA CATTCTAAAA TTTAGGGAAA
AGGACCAATG GTGCAAACAT TTTAGAGCTG ACAGTTGTGT GCCATATGCC
ATGATTCTGT TACAAATGAA CAGTATTCAG ATTCAAAATC AGTGTAAACA
CTGTGTGTGT GTGTGTGTGT GTGTGTGTGT GTGTGTGTGT GTATTTTACA
CAGCCATTTA AATATTAACC GCCTTTGAGT ATTAGGGGAA AAAACAGAAA
CTAAAAGCGA ATATATTTGT TCCTGAATCC TCCCACCAAA CCACTTTTTA
AATTAATTAT ATTTCCTGCC TTAGTGAGGA TCTTCCTATT CATCAAAGAT
AAAATACCAA ATATAATTTA CTCTCCTTCT CTACCTCACC CCCAATATTC
AACAATTCCC TATTTTATTT TGATTTTACT TCCTATTGTC TCTCAGTGCA
TCCTTACTAC TGGTTTCTGG CCTCAATGTC TCTTTCCATA AATACTTCCT
CCAGGGCTTC AGCAGTGTAT ATTGCAATCC ATGAGTGTGA TGCCCTTATA
AGCTGTACAG GCACAACCCA GGCAAACATA CACAATGACC ATAATCATAA
CAGTCATTAC TGGTGCCTTT ACTCTGGTTT TATCCATCCC CCAACAATCC
TTTAATCCTC CACTAGAGTT TATTCTTTTA AGTGAAAATC TGGATTCTTA
TCTCCCCTAG TATGTATCTC TTAGTGAATT TTTATATGAG ATAGTCATCA
CCATCACCTA TTCATCATCC CATTCCATCC ACTGAAGTTA TTTTACGCTA
TGGCCTTCAC AAATTTACTT TGAGTCCTTT AAATAGATTA AAAAGTTTCT
TGACTCTTTT TGTACATGTC ATTCTGTCCA AAGTGCAAAT TTCACTATTG
TCCACCCAAA GAATACTAAG TTTTCACCGT CATTTAAGCC TTTAATCTCA
GGATCTCACA ATAAATACAA TCACTCTTTC CTATATGCCT AATCTTCTGC
TTGAGCATAA TTTTAATGTG CTAACTATTC TGTAATTATA TATATATTTT
TAATTCAGCC ACTTCTCTCA CTAGAAAGTG AGTTTGTTGA AATCAGGGTA
AGTATATTTT ATGTTTGGAT GAATTCCCCA TCACAATACT TCACATGTAG
TTATCTAGTC ACCAATTTTT ATTGAAATTA ATTGTACATA TAATAAAACT
TTAATATAAA ATGTTTCTCT TGAGGGGAGA TTTTCTTTGT AAAACTATCC
TTCTGAGCTT TGTGATGTGA TGATTGTCTA ATGTCTGTTG CAAGATTAAG
GAAAATGTAT TTGAATGCAA ATGAACTTAC ACTGTCATAC CAAAAGTGTG
ATAATTTCTT GCTCCTGAAC TCACTCTCCC TACCTGCCTA TTAAAATCAG
AATACACAGA TCTGTATCTG TACAAAGAGA TGCCAAAAGA CTAC-3′
underlined nucleotides: hChr7 genomic primer sequence.
bold nucleotides: homologous extremity of the 213 bp
amplicon obtained with the primers MSP2#3-5.
The internal underlined nucleotides correspond to
the sequence of primer hChr7/4293490 S (hChr7#2).
Other amplicons obtained from HIV positive or
HIV-negative patients or from both using the
human chromosome 1 or human chromosome 7
primers described in sections C) and D) below.
C) Genomic human Chromosome 1 primers #3
(hChr1/14180006 S) and #4 (hChr1/14180401 AS)
PCR-amplified 396 bp amplicon (SEQ ID NO: 30):
Identifier: Amplicon hChr1/14180006-141a0401
5′-
CATAGCTTCC TAGTAAGTAG ACCAGCCTTC AGTCTGAGCC CTCCTCGGTC
CTTCCTCCCC AGTGCTGCTG GAGTAATCCT TCTAACACAA CAATGAAAGC
AGGTCACTGC GGCTCAAATG ATGTCAGCGG CTTTATCATC CATGTTGCCT
GGCTTTTCAC AGGCATGTCT TGCAGTGCAG CCTTATAACT CTCTCAACAC
AACTCTGTAT CCTCCTCATT CTTCATGCTT TTATAATGTC AAGCCATGTG
ACACTCCCTA AATATACCAT GTTTTCTCTT TTTCCTCCTC CCCCTCTCTC
ATTTGCAGCT TCCCATACTT ATCTTCCTAA ACACTACTCT TTTTGAAATG
TTTATTTCAA GGGTTTCTTA TCTTTTAAAC CATCTCAGAC TCCCCT-3′
underlined nucleotide: primer sequences.
bold nucleotide: homologous extremity of the 237 bp
amplicon obtained with primers MSP2#3-5.
D) Genomic human Chromosome 1 primers #3
(hChr1/14180006 S) and #5 (hChr1/14181093 AS)
PCR-amplified 1,088 bp amplicon (SEQ ID NO: 31):
Identifier: Amplicon hChr1/14180006-14181093
5′-
CATAGCTICC TAGTAAGTAG ACCAGCCTTC AGTCTGAGCC CTCCTCGGTC
CTTCCTCCCC AGTGCTGCTG GAGTAATCCT TCTAACACAA CAATGAAAGC
AGGTCACTGC GGCTCAAATG ATGTCAGCGG CTTTATCATC CATGTTGCCT
GGCTTTTCAC AGGCATGTCT TGCAGTGCAG CCTTATAACT CTCTCAACAC
AACTCTGTAT CCTCCTCATT CTTCATGCTT TTATAATGTC AAGCCATGTG
ACACTCCCTA AATATACCAT GTTTTCTCTT TTTCCTCCTC CCCCTCTCTC
ATTTGCAGCT TCCCATACTT ATCTTCCTAA ACACTACTCT TTTTGAAATG
TTTATTTCAA GGGTTTCTTA TCTTTTAAAC CATCTCAGAC TCCCCTGGGG
ATTACCCCTT TTCCTATGTT TTTATTGTAG CATCCTCACA AATTCACTTT
AGTTCCTTCG CATTCTGGTG TCGCTATATA TTAGTGGGAC TATGTCCCCA
TTAACCTGTT AGATCTCTTG AGAAAAGGGA CATGTCTTTT CATCTTGAGT
TCCCCAATAC TTAGTATTGT GCTTAGCATA TGCTAGGTGC TCAGTAAATA
TTTGATATGT GTGTGAACGA ATGAATCAAT CAATCAATAA CAAATGACAG
ACAAACTCCA ACCCCCAAAC CTAAAAAAAA AAAATCCAAA CTTTCCCCTT
GCTCTTAGTG TAGATACTGC TCATCAACAT AAGGCAAATT CTTCCTGCGC
GTCTCAATAC AGAGGAGGCG AGAACTCACA GAATCACAGA ATTAGAGCAC
TGGCTTTGGC ATGAGAACAC CCTGAGTTAA AATCTGGCTT CTGCTATTTA
TTAGCCACAT GACAGTGAAT CTCCTTGAGC TTCTGTTTTG TACAAACTTA
AGTTTGGCTT TGTGATCTTA TTCCTCTTTG GTGCATCTGT ACAACCCAAC
TGCTTATTCA TATGACACTG CTAAAACATG CCTTGCCTTC TCCCCCACTT
TTTTTTTTGG AGACAGAATC TCCCTTTGTC ACCCAGGCTG GAATTCAGTG
GCGTGATCTC GGCTCACTGC AACCTCCACC TCTCCAGT-3′
underlined nucleotides: primer sequences.
Bold nucleotides: homologous extremity of the 237 bp
amplicon obtained with the primers MSP2#3-5.
The sequence of the 396bp amplicon hChr1/14180006-
14180401 (C) is included in the larger size
amplicon 1,088 Amplicon hChr1/14180006-14181093.
The internal underlined nucleotides correspond to the
reverse-complement sequence ofprimer hChr1/14180401
AS (hChr1#4).
E) Genomic human Chromosome 7 primers #2
(hChr7/4293490 S) and #6 (hChr7/4294983 AS)
PCR-Amplified 1,494 bp amplicon (SEQ ID NO: 32):
Identifier: Amplicon hChr7/4293490-4294983
5′-
TCCTGCCTTA GTGAGGATCT TCCTATTCAT CAAAGATAAA ATACCAAATA
TAATTTACTC TCCTTCTCTA CCTCACCCCC AATATTCAAC AATTCCCTAT
TTTATTTTGA TTTTACTTCC TATTGTCTCT CAGTGCATCC TTACTACTGG
TTTCTGGCCT CAATGTCTCT TTCCATAAAT ACTTCCTCCA GGGCTTCAGC
AGTGTATATT GCAATCCATG AGTGTGATGC CCTTATAAGC TGTACAGGCA
CAACCCAGGC AAACATACAC AATGACCATA ATCATAACAG TCATTACTGG
TGCCTTTACT CTGGTTTTAT CCATCCCCCA ACAATCCTTT AATCCTCCAC
TAGAGTTTAT TCTTTTAAGT GAAAATCTGG ATTCTTATCT CCCCTAGTAT
GTATCTCTTA GTGAATTTTT ATATGAGATA GTCATCACCA TCACCTATTC
ATCATCCCAT TCCATCCACT GAAGTTATTT TACGCTATGG CCTTCACAAA
TTACTTTGAG TCCTTTAAAT AGATTAAAAA GTTTCTTGAC TCTTTTTGTA
CATGTCATTC TGTCCAAAGT GCAAATTTCA CTATTGTCCA CCCAAAGAAT
ACTAAGTTTT CACCGTCATT TAAGCCTTTA ATCTCAGGAT CTCACAATAA
ATACAATCAC TCTTTCCTAT ATGCCTAATC TTCTGCTTGA GCATAATTTT
AATGTGCTAA CTATTCTGTA ATTATATATA TATTTTTAAT TCAGCCACTT
CTCTCACTAG AAAGTGAGTT TGTTGAAATC AGGGTAAGTA TATTTTATGT
TTGGATGAAT TCCCCATCAC AATACTTCAC ATGTAGTTAT CTAGTCACCA
ATTTTTATTG AAATTAATTG TACATATAAT AAAACTTTAA TATAAAATGT
TTCTCTTGAG GGGAGATTTT CTTTGTAAAA CTATCCTTCT GAGCTTTGTG
ATGTGATGAT TGTCTAATGT CTGTTGCAAG ATTAAGGAAA ATGTATTTGA
ATGCAAATGA ACTTACACTG TCATACCAAA AGTGTGATAA TTTCTTGCTC
CTGAACTCAC TCTCCCTACC TGCCTATTAA AATCAGAATA CACAGATCTG
TATCTGTACA AAGAGATGCC AAAAGACTAC TTTCATGCTG CAACATGATT
ATGTGCCCCC AAAACCTGGA TATTTATAGT ATAGTATCCA GTATTTTCAA
TCTAAGCTGT ACTGGAGCCC GAAGCTAAAG GAAAATTAGT AATACTGATG
CTCCCTTTAT TTAAACTTTT AAGACTTTAT CATGGCATTA ATTTTGACTT
TTAAAAATAT TATCATTTTT TTTGGACCCC CTTAAATTTT GTTCCCGAGT
TGAATGCCTC ACTGGGACTT GGGTGAATGA ATGCTCACCC TAGTCCTAGA
TTAGGTACTC ATCTTAAATA CTGTTAGTTT GGGGTGGTTT TTTTTTTTTT
TTTTTTTTTT TTTTTGACAG AGCCTCACTC TGTTCTCCAG GCTG-3′
underlined nucleotides: hChr7 genomic primer sequence.
bold nucleotides: homologous sequence of the 213 bp
amplicon obtained with the primers
MSP2#2-5.
A part of the sequence 1,644 bp amplicon
hChr7/4292976-4294619 (B) is included in the
amplicon 1,494 bp Amplicon hChr7/4293490-4294983 (E).
The internal underlined nucleotides correspond to
the reverse-complement sequence of primer
hChr7/4294619 AS (hChr7#5).

APPENDIX 6
An amplicon of the 16s rDNA primers
(SEQ ID NOS: 24 and 25) is shown below.
The amplified DNA originated from the
red blood cells of an HIV-negative
subject passaged in HL60 cells. Similar
DNA is amplified from samples originating
from red 5 blood cells of HIV-positive
subjects. Amplicon (SEQ ID NO: 33) from
HIV-negative subject obtained using
primers (SEQ ID NOS: 24 and 25):
>T56-EMK-4-HIV-_Anae#2-5
wo/ primers seq. = 681 bp

5′-GCACCTGTATGTGAATTCCCGAAGGCACT
CCCGCATCTCTGCAGGATTCTCACTATGTCAA
GACCAGGTAAGGTTCTTCGCGTTGCATCGAAT
TAAACCACATGCTCCACCGCTTGTGCGGGCCC
CCGTCAATTCATTTGAGTTTTAACCTTGCGGC
CGTACTCCCCAGGCGGTCTACTTATCGCGTTA
ACTGCGCCACTAAAGTCTCAAGGACCCCAACG
GCTAGTAGACATCGTTTACGGCGTGGACTACC
AGGGTATCTAATCCTGTTTGCTACCCACGCTT
TCGAATCTCAGTGTCAATATTATGCCAGGAAG
CTGCCTTCGCCATCGGCATTCCTCCAGATCTC
TACGCATTTCACCGCTACACCTCGAATTCTA
CTTCCCTCTCACATATTCTAGCACCACCAGTA
TCACATGCAGTTCCCAGGTTAAGCCCGGGGAT
TTCACATGTGACTTAATGAGCCACCTACACTC
GCTTTACGCCCAGTAATTCCGATTAACGCTCG
CACCCTCTGTATTACCGCGGCTGCTGGCACAG
AGTTAGCCGGTGCTTATTCTGCAGGTAACGTC
TAATCTAATGGGTATTAACCATTAGCCTCTCC
TCCCTGCTTAAAGTGCTTTACAACCAAAAGGC
CTTCTTCACACACGCGGCATGGCTGGAT CA
GGGTTGCCCCCCATT-3′

What is claimed is:

1. A primer for detecting an agent that is associated with human red blood cells, selected from the group consisting SEQ ID NOS: 1-25, or one or more nucleic acids effective for use in amplifying at least twenty consecutive nucleotides of the same DNA as said primers SEQ ID NOS: 1-25.

2. The primer according to claim 1, further comprising a second primer selected from the group consisting of SEQ ID NOS: 1-25 a primer effective for use in amplifying at least twenty consecutive nucleotides of the same DNA as said primers selected from the group consisting of SEQ ID NOS: 1-25, wherein the primer and the second primer comprise a sense primer and an antisense primer.

3. The primer according to claim 1, wherein the primer and the second primer comprises a nucleic acid sequence selected from the group consisting of (SEQ ID NOS: 3 and 4) and (SEQ ID NOS: 5 and 6) or a pair of primers effective for use in amplifying at least twenty consecutive nucleotides of the same DNA as said primers selected from the group consisting of (SEQ ID NOS: 3 and 4) and (SEQ ID NOS: 5 and 6).

4. The primer according to claim 1, comprising a pair of primers comprising a sense primer and an antisense primer selected from the group consisting of (SEQ ID NOS: 7-14) and (SEQ ID NOS: 15-23) or a pair of primers effective for use in amplifying at least twenty consecutive nucleotides of the same DNA as said primers selected from the group consisting of (SEQ ID NOS: 7-14) and (SEQ ID NOS: 15-23).

5. Isolated or purified DNA having a DNA sequence and a DNA length that is produced by amplifying DNA using a polymerase chain reaction, using a pair of primers according to claim 1.

6. The isolated or purified DNA according to claim 5, whereon the primers are selected from the group consisting of SEQ ID NOS: 3-6.

7. The isolated or purified DNA according to claim 5, whereon the primers are selected from the group consisting of SEQ ID NOS: 7-23.

8. The isolated or purified DNA according to claim 5, that comprises a DNA sequence or fragment thereof described by Appendix 5 (SEQ ID NO: 26).

9. The isolated or purified DNA according to claim 5, that comprises a DNA sequence or fragment thereof described by Appendix 5 (SEQ ID NO: 27).

10. The isolated or purified DNA according to claim 5, that comprises a DNA sequence or fragment thereof described by Appendix 5 (SEQ ID NO: 28).

11. The isolated or purified DNA 8 according to claim 5, that comprises a DNA sequence or fragment thereof described by Appendix 5 (SEQ ID NO: 29).

12. The isolated or purified DNA according to claim that comprises a DNA sequence or fragment thereof described by Appendix 5 (SEQ ID NO: 30).

13. The isolated or purified DNA according to claim 5, that comprises a DNA sequence or fragment thereof described by Appendix 5 (SEQ ID NO: 31).

14. The isolated or purified DNA according to claim 5, that comprises a DNA sequence or fragment thereof described by Appendix 5 (SEQ ID NO: 32).

15. A method for detecting an agent that is associated with human red blood cells, comprising:
performing a polymerase chain reaction amplification of DNA from the human red blood cells using at least one primer selected from the group consisting of SEQ ID NOS: 1-25 or a primer effective for use in amplifying at least twenty consecutive nucleotides of the same DNA as said primers SEQ ID NOS: 1-25; and
detecting a selectively produced amplicon from the polymerase chain reaction amplification.

16. The method according to claim 15, wherein the at least one primer comprises a pair of primers selected from the group consisting of (SEQ ID NOS: 3 and 4) and (SEQ ID NOS: 5 and 6) or a pair of primers effective for use in amplifying at least twenty consecutive nucleotides of the same DNA as said primers selected from the group consisting of (SEQ ID NOS: 3 and 4) and (SEQ ID NOS: 5 and 6).

17. The method according to claim 15, wherein the at least one primer comprises a pair of primers selected from the group consisting of (SEQ ID NOS: 7-14) and (SEQ ID NOS: 15-23) or a pair of primers effective for use in amplifying at least twenty consecutive nucleotides of the same DNA as said primers selected from the group consisting of (SEQ ID NOS: 7-14) and (SEQ ID NOS: 15-23).

18. The method according to claim 15, wherein the at least one primer comprises a pair of primers (SEQ ID NOS: 24 and 25) or a pair of primers effective for use in amplifying at least twenty consecutive nucleotides of the same DNA as said primers (SEQ ID NOS: 24 and 25).

19. The method according to claim 15, wherein the selectively produced amplicon comprises a DNA sequence or fragment thereof described by SEQ ID NOS: 26-32.

20. The method according to claim 15, wherein the selectively produced amplicon comprises a DNA sequence or fragment thereof described by SEQ ID NO: 33.


SEQUENCE LISTING (33 sequences)

SEQ ID NO: 1 - DNA, 20 bp, Artificial Sequence
  Primer
gcaacgcgaaaaaccttacc

SEQ ID NO: 2 - DNA, 18 bp, Artificial Sequence
  Primer
gacgggcagtgtgtacaa

SEQ ID NO: 3 - DNA, 18 bp, Artificial Sequence
  Primer
gcctacagattaaaggct

SEQ ID NO: 4 - DNA, 20 bp, Artificial Sequence
  Primer
atcatartcaccatcaccta

SEQ ID NO: 5 - DNA, 17 bp, Artificial Sequence
  Primer
cytacagagtgaaggct

SEQ ID NO: 6 - DNA, 20 bp, Artificial Sequence
  Primer
atcatartcaccatcaccta

SEQ ID NO: 7 - DNA, 20 bp, Artificial Sequence
  Primer
ccttacactcagccagacat

SEQ ID NO: 8 - DNA, 20 bp, Artificial Sequence
  Primer
ccaggtgtgtggcttataca

SEQ ID NO: 9 - DNA, 25 bp, Artificial Sequence
  Primer
catagcttcctagtaagtagaccag

SEQ ID NO: 10 - DNA, 18 bp, Artificial Sequence
  Primer
aggggagtctgagatggt

SEQ ID NO: 11 - DNA, 18 bp, Artificial Sequence
  Primer
actggagaggtggaggtt

SEQ ID NO: 12 - DNA, 21 bp, Artificial Sequence
  Primer
ggtaattcctatgtgcgaggt

SEQ ID NO: 13 - DNA, 21 bp, Artificial Sequence
  Primer
tcaaaagacagtggtgactct

SEQ ID NO: 14 - DNA, 20 bp, Artificial Sequence
  Primer
atgtctggctgagtgtaagg

SEQ ID NO: 15 - DNA, 24 bp, Artificial Sequence
  Primer
atgtagttgagcagttttgaatga

SEQ ID NO: 16 - DNA, 20 bp, Artificial Sequence
  Primer
tcctgccttagtgaggatct

SEQ ID NO: 17 - DNA, 23 bp, Artificial Sequence
  Primer
atgaataggtgatggtgatgact

SEQ ID NO: 18 - DNA, 24 bp, Artificial Sequence
  Primer
ccgtcatttaagcctttaatctca

SEQ ID NO: 19 - DNA, 24 bp, Artificial Sequence
  Primer
gtagtcttttggcatctctttgta

SEQ ID NO: 20 - DNA, 19 bp, Artificial Sequence
  Primer
cagcctggagaacagagtg

SEQ ID NO: 21 - DNA, 24 bp, Artificial Sequence
  Primer
gatctagcagttcataggaaggaa

SEQ ID NO: 22 - DNA, 20 bp, Artificial Sequence
  Primer
ggctgaaacaatggggtttt

SEQ ID NO: 23 - DNA, 20 bp, Artificial Sequence
  Primer
tttagtagacccctcgacca

SEQ ID NO: 24 - DNA, 17 bp, Artificial Sequence
  Primer
ctgacgacagccatgca

SEQ ID NO: 25 - DNA, 20 bp, Artificial Sequence
  Primer
gcagtggggaatattggaca

SEQ ID NO: 26 - DNA, 237 bp, Artificial Sequence
  237 bp amplicon generated with MSP2 primer #3 and MSP2 primer #5 by PCR
atcatagtcaccatcacctaccagctgtataagccacacacctgggagtcctcctagcctttttcctcctcctctcatcctccatatcccattgaccgtcagggcctactgagtctacactccaattttcttttaaatctatccccactgccactgtcctagtctaaggcaataccatctggtcacccagatcattccatagcttcctagtaagtagaccagccttcactctgtaag

SEQ ID NO: 27 - DNA, 213 bp, Artificial Sequence
  213 bp amplicon generated with MSP2 primer #2 and MSP2 primer #5 by PCR
gcctacagattaaaggcttaaatgacggtgaaaacttagtattctttgggtggacaatagtgaaatttgcactttggacagaatgacatgtacaaaaagagtcaagaaactttttaatctatttaaaggactcaaagtaatttgtgaaggccatagcgtaaaataacttcagtggatggaatgggatgatgaataggtgatggtgactatgat

SEQ ID NO: 28 - DNA, 546 bp, Artificial Sequence
  546 bp amplicon of genomic human chromosome 1 amplified by primers hChr1/14179308 S and hChr1/14179853 AS
ccttacactcagccagacatatatttgtgttttgttatccatgtgcacagagactttggcattctgggtgaaggaagaaagaagagaatatacatggaaacccaggggtaagagaaaaggacaacagagaatgtggcatggggaatgctctgctgggtcacattgaatggttctgaaccactgtggaaaaaaaggagttagaaagaatcagatgccgaaggagccaattttcacaatactccgagactcagggcaaaagcagccttgttctagtagcctatgggtaaaagaagacacagaactgaggggaggacttttcccctgagtccaccacaaaccgccatggagctgaggcagcctgaagtctcaggggcatgggagggatttgccttttggatttctccaatgggatgtcttacaggcacttcatatttagcagatccaaaacttaactcagatactcctcttgccatatctgttcctcttgctgtgttcctgaccatgattatcaccatcacctaccagctgtataagccacacacctgg

SEQ ID NO: 29 - DNA, 1644 bp, Artificial Sequence
  1644 bp amplicon of genomic human chromosome 7 amplified by primers hChr7/4292976 S and hChr7/4294619 AS
atgtagttgagcagttttgaatgagtttcttaatcctgagttctagtttaagaaaatattaaaaataaaaaattatgtcaccaactaaatttttactgcagataatcataagttggttagattggaccttcattgtgaaatgcagtaactttggtttaagcaatatccaaaaccagaaattggtcgaggggtctactaaattccgttttcttttgttctaaacaattaaacattctaaaatttagggaaaaggaccaatggtgcaaacattttagagctgacagttgtgtgccatatgccatgattctgttacaaatgaacagtattcagattcaaaatcagtgtaaacactgtgtgtgtgtgtgtgtgtgtgtgtgtgtgtgtgtgtgtgtattttacacagccatttaaatattaaccgcctttgagtattaggggaaaaaacagaaactaaaagcgaatatatttgttcctgaatcctcccaccaaaccactttttaaattaattatatttcctgccttagtgaggatcttcctattcatcaaagataaaataccaaatataatttactctccttctctacctcacccccaatattcaacaattccctattttattttgattttacttcctattgtctctcagtgcatccttactactggtttctggcctcaatgtctctttccataaatacttcctccagggcttcagcagtgtatattgcaatccatgagtgtgatgcccttataagctgtacaggcacaacccaggcaaacatacacaatgaccataatcataacagtcattactggtgcctttactctggttttatccatcccccaacaatcctttaatcctccactagagtttattcttttaagtgaaaatctggattcttatctcccctagtatgtatctcttagtgaatttttatatgagatagtcatcaccatcacctattcatcatcccattccatccactgaagttattttacgctatggccttcacaaatttactttgagtcctttaaatagattaaaaagtttcttgactctttttgtacatgtcattctgtccaaagtgcaaatttcactattgtccacccaaagaatactaagttttcaccgtcatttaagcctttaatctcaggatctcacaataaatacaatcactctttcctatatgcctaatcttctgcttgagcataattttaatgtgctaactattctgtaattatatatatatttttaattcagccacttctctcactagaaagtgagtttgttgaaatcagggtaagtatattttatgtttggatgaattccccatcacaatacttcacatgtagttatctagtcaccaatttttattgaaattaattgtacatataataaaactttaatataaaatgtttctcttgaggggagattttctttgtaaaactatccttctgagctttgtgatgtgatgattgtctaatgtctgttgcaagattaaggaaaatgtatttgaatgcaaatgaacttacactgtcataccaaaagtgtgataatttcttgctcctgaactcactctccctacctgcctattaaaatcagaatacacagatctgtatctgtacaaagagatgccaaaagactac

SEQ ID NO: 30 - DNA, 396 bp, Artificial Sequence
  396 bp amplicon of genomic human chromosome 1 amplified by primers hChr1/14180006 S and hChr1/14180401 AS
catagcttcctagtaagtagaccagccttcagtctgagccctcctcggtccttcctccccagtgctgctggagtaatccttctaacacaacaatgaaagcaggtcactgcggctcaaatgatgtcagcggctttatcatccatgttgcctggcttttcacaggcatgtcttgcagtgcagccttataactctctcaacacaactctgtatcctcctcattcttcatgcttttataatgtcaagccatgtgacactccctaaatataccatgttttctctttttcctcctccccctctctcatttgcagcttcccatacttatcttcctaaacactactctttttgaaatgtttatttcaagggtttcttatcttttaaaccatctcagactcccct

SEQ ID NO: 31 - DNA, 1088 bp, Artificial Sequence
  1088 bp amplicon of genomic human chromosome 1 amplified by primers hChr1/14180006 S and hChr1/14181093 AS
catagcttcctagtaagtagaccagccttcagtctgagccctcctcggtccttcctccccagtgctgctggagtaatccttctaacacaacaatgaaagcaggtcactgcggctcaaatgatgtcagcggctttatcatccatgttgcctggcttttcacaggcatgtcttgcagtgcagccttataactctctcaacacaactctgtatcctcctcattcttcatgcttttataatgtcaagccatgtgacactccctaaatataccatgttttctctttttcctcctccccctctctcatttgcagcttcccatacttatcttcctaaacactactctttttgaaatgtttatttcaagggtttcttatcttttaaaccatctcagactcccctggggattaccccttttcctatgtttttattgtagcatcctcacaaattcactttagttccttcgcattctggtgtcgctatatattagtgggactatgtccccattaacctgttagatctcttgagaaaagggacatgtcttttcatcttgagttccccaatacttagtattgtgcttagcatatgctaggtgctcagtaaatatttgatatgtgtgtgaacgaatgaatcaatcaatcaataacaaatgacagacaaactccaacccccaaacctaaaaaaaaaaaatccaaactttccccttgctcttagtgtagatactgctcatcaacataaggcaaattcttcctgcgcgtctcaatacagaggaggcgagaactcacagaatcacagaattagagcactggctttggcatgagaacaccctgagttaaaatctggcttctgctatttattagccacatgacagtgaatctccttgagcttctgttttgtacaaacttaagtttggctttgtgatcttattcctctttggtgcatctgtacaacccaactgcttattcatatgacactgctaaaacatgccttgccttctcccccacttttttttttggagacagaatctccctttgtcacccaggctggaattcagtggcgtgatctcggctcactgcaacctccacctctccagt

SEQ ID NO: 32 - DNA, 1494 bp, Artificial Sequence
  1494 bp amplicon of genomic human chromosome 7 amplified by primers hChr7/4293490 S and hChr7/4294983 AS
tcctgccttagtgaggatcttcctattcatcaaagataaaataccaaatataatttactctccttctctacctcacccccaatattcaacaattccctattttattttgattttacttcctattgtctctcagtgcatccttactactggtttctggcctcaatgtctctttccataaatacttcctccagggcttcagcagtgtatattgcaatccatgagtgtgatgcccttataagctgtacaggcacaacccaggcaaacatacacaatgaccataatcataacagtcattactggtgcctttactctggttttatccatcccccaacaatcctttaatcctccactagagtttattcttttaagtgaaaatctggattcttatctcccctagtatgtatctcttagtgaatttttatatgagatagtcatcaccatcacctattcatcatcccattccatccactgaagttattttacgctatggccttcacaaattactttgagtcctttaaatagattaaaaagtttcttgactctttttgtacatgtcattctgtccaaagtgcaaatttcactattgtccacccaaagaatactaagttttcaccgtcatttaagcctttaatctcaggatctcacaataaatacaatcactctttcctatatgcctaatcttctgcttgagcataattttaatgtgctaactattctgtaattatatatatatttttaattcagccacttctctcactagaaagtgagtttgttgaaatcagggtaagtatattttatgtttggatgaattccccatcacaatacttcacatgtagttatctagtcaccaatttttattgaaattaattgtacatataataaaactttaatataaaatgtttctcttgaggggagattttctttgtaaaactatccttctgagctttgtgatgtgatgattgtctaatgtctgttgcaagattaaggaaaatgtatttgaatgcaaatgaacttacactgtcataccaaaagtgtgataatttcttgctcctgaactcactctccctacctgcctattaaaatcagaatacacagatctgtatctgtacaaagagatgccaaaagactactttcatgctgcaacatgattatgtgcccccaaaacctggatatttatagtatagtatccagtattttcaatctaagctgtactggagcccgaagctaaaggaaaattagtaatactgatgctccctttatttaaacttttaagactttatcatggcattaattttgacttttaaaaatattatcattttttttggacccccttaaattttgttcccgagttgaatgcctcactgggacttgggtgaatgaatgctcaccctagtcctagattaggtactcatcttaaatactgttagtttggggtggttttttttttttttttttttttttttttgacagagcctcactctgttctccaggctg

SEQ ID NO: 33 - DNA, 681 bp, Artificial Sequence
  681 bp amplicon of HIV-negative subject amplified by primers of SEQ ID NOs 24 and 25
gcacctgtatgtgaattcccgaaggcactcccgcatctctgcaggattctcactatgtcaagaccaggtaaggttcttcgcgttgcatcgaattaaaccacatgctccaccgcttgtgcgggcccccgtcaattcatttgagttttaaccttgcggccgtactccccaggcggtctacttatcgcgttaactgcgccactaaagtctcaaggaccccaacggctagtagacatcgtttacggcgtggactaccagggtatctaatcctgtttgctacccacgctttcgaatctcagtgtcaatattatgccaggaagctgccttcgccatcggcattcctccagatctctacgcatttcaccgctacacctggaattctacttccctctcacatattctagcaccaccagtatcacatgcagttcccaggttaagcccggggatttcacatgtgacttaatgagccacctacactcgctttacgcccagtaattccgattaacgctcgcaccctctgtattaccgcggctgctggcacagagttagccggtgcttattctgcaggtaacgtctaatctaatgggtattaaccattagcctctcctccctgcttaaagtgctttacaaccaaaaggccttcttcacacacgcggcatggctggatcagggttgccccccatt